(19) United States
(12) Patent Application Publication
(10) Pub. No.: US 2016/0318836 A1
(43) Pub. Date: Nov. 3, 2016

(54) PROCESS FOR THE PRODUCTION OF 2,6-DIMETHYLHEPT-5-ENAL BY BAEYER-VILLIGER OXIDATION

(71) Applicant: GIVAUDAN SA, Vernier (CH)

(72) Inventors: Alan John CHALK, Lewes, DE (US); Andreas GOEKE, Winterthur (CH); Martin Alan LOVCHIK, Duebendorf (CH)

(73) Assignee: GIVAUDAN SA, Vernier (CH)

(21) Appl. No.: 15/103,592

(22) PCT Filed: Jan. 22, 2015
(86) PCT No.: PCT/EP2015/051207

§ 371 Date: Jun. 10, 2016

Related U.S. Application Data
(60) Provisional application No. 61/930,764, filed on Jan. 23, 2014.

(51) Int. Cl. C07C 45/54; C07C 67/44

(52) U.S. Cl. CPC C07C 45/54; C07C 67/44

(57) ABSTRACT

Disclosed is a process for the production of 2,6-dimethylhept-5-enal by Baeyer-Villiger oxidation of 3,7-dimethylocta-2,6-dienal in the presence of aqueous H₂O₂ and SeO₂, followed by hydrolysis.

The present invention is concerned with a novel process for the production of 2,6-dimethylhept-5-enal, also known as Melonal, which is an important, aldehydic, melon-like odorant for the fragrance industry. In particular, the present invention relates to a process for the synthesis Melonal by selenium dioxide (SeO₂) catalyzed Bayer-Villiger reaction of (E/Z)-3,7-dimethylocta-2,6-dienal, also known as Citral, via the intermediate 2,6-dimethylhepta-1,5-dien-1-yl formate.

Up to now 2,6-dimethyl-5-heptenal is industrially produced by Darzens reaction from 6-methyl-hept-5-en-2-one. This reaction requires alkylation with chloro acetic esters to glycidesters followed by hydrolysis and decarboxylation leading to the aldehyde. This approach is not atom economic as an alcohol (ROH), a chloride salt, as well as carbon dioxide is eliminated from the reactants.

Some years ago Corma at al (J. Catal. 2005, 234, 96) reported a heterogeneous Baeyer-Villiger reaction of citral (1) applying zeolites and other mosoprous materials followed by hydrolysis of the intermediate formate ester. Unfortunately not only 2,6-dimethylhepta-1,5-dien-1-yl formate (2), but also olfactory disturbing side products are generated, such as 6-methylhept-5-en-2-one, citral-6,7-epoxide, citral-2,3-epoxide and various cyclization products of citral, as shown Scheme 1.

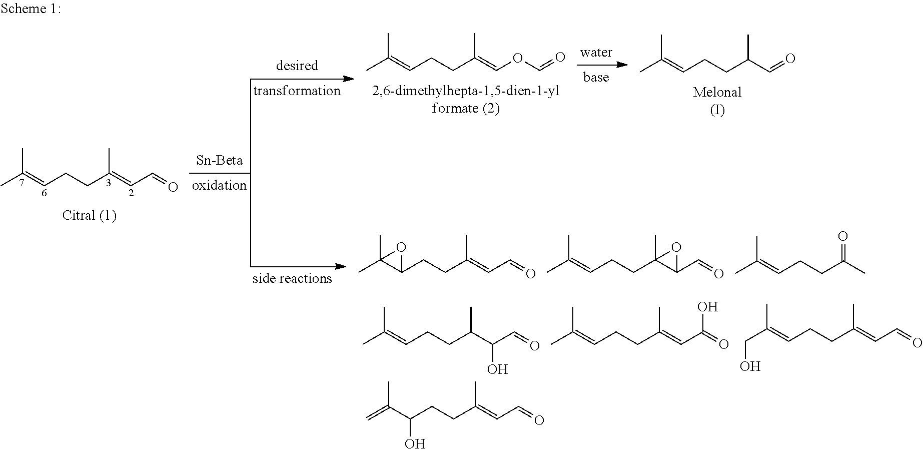

Furthermore, such heterogeneous catalysts need to be prepared in a separate process. Accordingly, there remains a need for a simple and cheap process for the production of Melonal in an olfactory pure form.

Surprisingly, inventors found that Melonal can be prepared starting from citral by selenium dioxide (SeO₂) catalyzed Bayer-Villiger reaction in the presence of aqueous H₂O₂ via the intermediate 2,6-dimethylhepta-1,5-dien-1-yl formate. Surprisingly, the use of SeO₂ in catalytic amounts resulted in a high purity of the reaction product.

Thus there is provided in a first aspect a process comprising the oxidation of 3,7-dimethylocta-2,6-dienal (1) in the presence of aqueous H₂O₂ (e.g. an aqueous solution containing between 25-70% H₂O₂), wherein the process is catalyzed by SeO₂, resulting in 2,6-dimethylhepta-1,5-dien-1-yl formate (2). In a subsequent step the formate (2) is hydrolyzed to melonal (I).

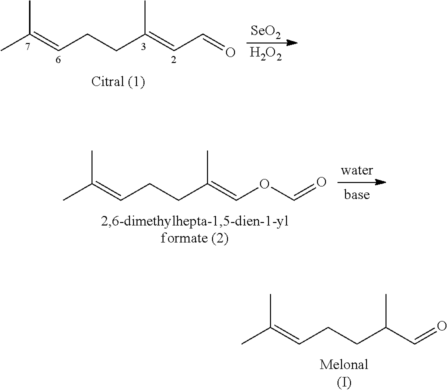

The reaction is catalyzed by catalytic amounts of selenium dioxide (SeO₂) at loadings of 0.01 to 50 mol %, preferentially at 0.1 to 5 mol % and even more preferred at 0.5 to 2 mol %. By using the homogenous catalyst SeO₂ it was possible to produce 2,6-dimethylhepta-1,5-dien-1-yl formate (2) without the presence of citral-6,7-epoxide and citral-2,3-epoxide.

Thus there is provided in a further aspect a process comprising the oxidation of 3,7-dimethylocta-2,6-dienal (1) in the presence of aqueous H₂O₂, wherein the process is catalyzed by SeO₂, resulting in a crude product which is essentially free of citral-6,7-epoxide and citral-2,3-epoxide.

By “essentially free” is meant, that the resulting product contains less than 5% by weight of citral-6,7-epoxide and less than 5% by weight of citral-2,3-epoxide. In one embodiment the resulting product contains less than 3% by weight (e.g., 1 weight % or less) of citral-6,7-epoxide and less than 3% by weight (e.g., 1 weight % or less) of citral-2,3-epoxide.

The Baeyer-Villiger reaction can be carried out at 100% conversion, but it is sometimes preferred to run the reaction at lower conversion, preferentially from 10-50% or more preferred at 20-40%, and to recycle remaining citral.

The reaction can be carried out in different solvents, protic or aprotic, miscible or not miscible with water, preferably miscible with water and also under heterogeneous and phase-transfer conditions. In one embodiment the reaction is carried out in a protic solvent (e.g. water).

Further examples of protic solvents include methanol, ethanol, tert-amyl alcohol and tert-butanol. Examples of aprotic solvents include acetone, butanone, tetrahydrofuran and ethyl acetate.

Similarly, the reactions may be carried out at different temperatures, preferably from 0° C.-200° C., e.g. up to 150° C., preferably from 20° C.-50° C., depending on the solvent used.

The invention is now further described with reference to the following non-limiting examples. These examples are for the purpose of illustration only and it is understood that variations and modifications can be made by one skilled in the art.

All products described in the examples were obtained starting from (E/Z)-3,7-dimethylocta-2,6-dienal (1). The first reaction product of the Baeyer-Villiger reaction, 2,6-dimethylhepta-1,5-dien-1-yl formate (2), is a mixture of E/Z isomers.

The reported NMR spectra were measured in CDCl₃ at 400 MHz if not otherwise stated; chemical shifts (8) are reported in ppm downfield from TMS; coupling constants J in Hz. The GC/MS analyses were run using a ZB-5 column, if not stated otherwise. All purified products were purified by distillation in vacuo and isolated as colorless oils, the purity was confirmed by GC/MS. Samples for olfactory evaluation were purified by rectification over a distillation column with Sulzer packing.

### EXAMPLE 1

The reactor was charged with (1) (114.0 g, 749 mmol), selenium dioxide (0.6 g, 5 mmol) and acetone (250 ml). The solution was stirred at room temperature and 30% aqueous hydrogen peroxide (71.5 g, 654 mmol) was added drop wise over 30 minutes. The reaction mixture was then stirred at room temperature for 16 hours.

Some of the acetone was removed by distillation until the reaction mixture became cloudy. Water (100 ml) and tert-butylmethyl ether (100 ml) was added and the layers were separated. The aqueous layer was extracted three times with tert-butylmethyl ether. The organic layers were combined and washed with water and brine. GC analysis showed a conversion of 40%.

The ether solution was placed in a reactor and cooled to 0-5° C. Aqueous 10% NaOH (150.0 g) was added drop wise over 30 minutes. After stirring at room temperature for additional 30 minutes, the layers were separated and the organic layer was washed two times with water (100 ml) and brine. The solution was concentrated in vacuo. The crude product (90.0 g) was flash distilled to give a colorless oil (76.0 g) with the following composition:

| Product | % | Weight (g) | Mol. |
|---|---|---|---|
| Melonal (I) | 37 | 28.4 | 0.20 |
| Citral (1) | 58 | 44.5 | 0.26 |
| Residue | | 11.0 | |

$$Yield of Melonal ( I ) \text{:} \frac{200 mmol \times 100}{749 mmol - 260 mmol} = 41 \%$$

### Olfactive Distillation:

A repeat experiment gave a crude weight of 578 g which was fractionally distilled at 10 mm (Hg).

| | | | | Melonal | | Citral |
|---|---|---|---|---|---|---|
| Fr. | Head (° C.) | Pot (° C.) | Weight (g) | (I) (g) | (I) (%) | (1) (%) |
| 1 | 49 | 75 | 2.7 | 2.6 | 95.3 | 0.6 |
| 2 | 64 | 82 | 107.6 | 106.6 | 99.1 | 0.8 |
| 3 | 67 | 83 | 46.6 | 46.1 | 99.0 | 1.0 |
| 4 | 65 | 95 | 67.9 | 66.7 | 98.3 | 1.3 |
| 5 | 60 | 101 | 16.7 | 12.4 | 74.3 | 1.7 |
| 6 | 65 | 105 | 19.4 | 6.0 | 30.7 | 65 |

Fraction 1, 5 and 6 were combined with the pot residue (304.5 g, consisting largely of citral) and recycled.

Fractions 2, 3 and 4=222.1 g was olfactively pure grade of melonal.

$$Weight yield \text{:} \frac{222.1 \times 100}{578.0} = 38 \%$$

¹H NMR: δ 9.58 (d, J=2.02, 1H); 5.05 (m, 1H); 2.31 (m, 1H); 2.00 (m, 2H); 1.72 (m, 1H); 1.65 (s, 3H); 1.56 (s, 3H); 1.36 (m, 1H); 1.06 (d, J=6.82, 3H). ¹³C NMR: δ 204.95 (d), 132.52 (s), 123.37, 45.69 (2d), 30.56 (t), 25.61 (q), 25.26 (t), 17.63, 13.15 (2q).

MS: 140 (10, W), 83 (12), 82 (100), 69 (29), 67 (48), 56 (12), 55 (19), 41 (48), 39 (18), 29 (15).

### EXAMPLE 2

### a) 2,6-dimethylhepta-1,5-dien-1-yl formate (2)

The reactor was charged with (1) (20.0 g, 131 mmol), water (50 ml) and selenium dioxide (1.0 g, 9 mmol). The mixture was heated to 40° C. and hydrogen peroxide 30% (29.8 g, 262 mmol) was added drop wise over 60 minutes. The reaction temperature rose to 55° C. and stirring was continued for one hour keeping the temperature at 40° C. After cooling of the reaction mixture to room temperature, the layers were separated and the aqueous layer was extracted two times with hexane (100 ml). The organic layers were combined, washed with saturated aqueous Na₂S₂O₃ (50 ml), water (50 ml) and brine (50 ml). Crude (2) (10.1 g) was obtained upon concentration of the organic solution having the following composition:

| Product | % | Weight (g) | Mmol. |
|---|---|---|---|
| 2,6-dimethylhepta-1,5-dien-1-yl formate (2) | 35 | 3.5 | 21 |
| Citral (1) | 65 | 6.6 | 43 |

$$Yield of ( 2 ) ( theory ) \text{:} \frac{21 mmol \times 100}{131 mmol - 43 mmol} = 24 \%$$

¹H NMR: δ 8.05 (d, J=0.51, 1H); 8.03 (d, J=0.50, 1H); 6.98 (m, 1H); 6.94 (m, 1H); 5.13-5.04 (m, 2H); 2.20-1.97 (m, 8H); 1.70 (d, J=1.52, 4H); 1.69 (m, 6H); 1.66 (d, 1.52; 2H); 1.61 (m, 6H). ¹³C NMR: δ 158.1 (2d), 132.2, 132.1 (2s), 128.9, 128.6 (2d), 123.6 (2s), 123.4, 123.3 (2d), 34.0, 26.1, 25.7 (4t), 26.1, 25.7, 25.6 (6q).

MS: 122 (20, M⁺ —CH₂O₂), 81 (14), 71 (14), 70 (11), 69 (100), 43 (15), 41 (53), 39 (15), 29 (14), 27 (88).

### b) 2,6-dimethylhept-5-enal (I)

The crude product of step a) (40.0 g, 90%, 214 mmol) was dissolved in diethyl ether (200 ml) and NaOH 10% (120.0 g, 30 mmol) was added. The mixture was stirred for 16 hours at room temperature. The layers were separated and the aqueous layer was extracted with pentane (100 ml). The organic layers were combined and washed with 10% acetic acid (50 ml), water (50 ml) and brine (50 ml). The organic solution was concentrated in vacuo and the crude product (I) (32.0 g) was flash distilled to give a colorless oil (26.4 g) with the following composition:

| Product | % | Weight (g) | Mmol. |
|---|---|---|---|
| Melonal (I) | 97.2 | 27.2 | 189 |
| Residue | | 6.0 | |

$$Yield of Melonal ( I ) \text{:} \frac{189 mmol \times 100}{214 mmol} = 88 \%$$

1. A process for the production of 2,6-dimethylhept-5-enal by Baeyer-Villiger oxidation of 3,7-dimethylocta-2,6-dienal in the presence of aqueous H₂O₂ and SeO₂, resulting in 2,6-dimethylhepta-1,5-dien-1-yl formate, followed by hydrolysis.

2. A process according to claim 1 wherein the process is carried out in a protic or aprotic solvent.

3. A process according to claim 2 wherein the solvent is selected from water, acetone, tert-butanol, and tert-amyl alcohol, or mixtures thereof.

4. A process according to claim 1, wherein the process is carried out from 0° C. to 200° C.

5. A process according to claim 1 wherein, 2,6-dimethylhepta-1,5-dien-1-yl formate is obtained which is essentially free of citral-6,7-epoxid and citral-2,3-epoxid.

6. A process according to claim 1 wherein the reaction is carried out at a conversion rate of citral from 10-100%.
